(19) United States
(12) Patent Application Publication
(10) Pub. No.: US 2013/0266524 A1
(43) Pub. Date: Oct. 10, 2013

(54) Oral Compositions

(71) Applicants: Ben Gu, East Brunswick, NJ (US); Mahmoud Hassan, Somerset, NJ (US)

(72) Inventors: Ben Gu, East Brunswick, NJ (US); Mahmoud Hassan, Somerset, NJ (US)

(73) Assignee: Cologate-Palmolive Company, New York, NY (US)

(21) Appl. No.: 13/993,720

(22) PCT Filed: Dec. 6, 2011
(86) PCT No.: PCT/US11/63367

§ 371 Date: Jun. 13, 2013

Related U.S. Application Data
(60) Provisional application No. 61/422,493, filed on Dec. 13, 2010.

(51) Int. Cl. A61K 8/81; A61K 8/73; A61Q 11/00

(52) U.S. Cl. CPC A61K 8/8176; A61Q 11/00; A61K 8/731
USPC 424/58; 424/49

(57) ABSTRACT

Described herein, are films, compositions containing the films and methods of preparing a dentifrice comprising the films, wherein the films contain a low solubility flavorant.

Some embodiments of the present invention relate to methods of making dentifrice products comprising hydratable, polymer matrix films.

### BACKGROUND

Liquid, gel and semi-solid oral care products which comprise hydratable, polymer matrix films containing low solubility flavorants such as menthol are known. See for example US2008/0014224A1 to Boyd et al. Hydratable, polymer matrix film containing low solubility flavorants such as menthol are prepared and added into toothpaste to generate an aesthetic effect as well as to provide the benefit of a flavor and/or cooling sensation or signal. The hydratable, polymer matrix film, typically in the form of flakes or small sized pieces cut from larger manufactured films, is maintained in the product when stored. Upon use, the films typically degrade by chemical or physical disruption, thereby releasing the active or functional material into the surrounding environment. In this manner, the films provide an opportunity for localized release of a high concentration of active materials, such as for example zinc oxide, near a target surface. In addition, low solubility flavorant in the film is also released. The low solubility flavorants such as menthol in the films provide an extended flavor experience to the user compared to that which occurs when compositions in which the flavor is only in the toothpaste base are used. By including flavorants in the films, flavor may be released from films during and immediately after use, providing a flavor experience that continues after performance of the oral care process such as brushing or rinsing is completed. This extended experience can be pleasing.

Conventional methods of manufacturing hydratable, polymer matrix films that contain low solubility flavorants such as menthol comprise the step of incorporating menthol into the slurry that is then used to form the film. The low solubility flavorant is thereby dispersed throughout the slurry which is used to make the film. After the film is formed, it is typically often cut into flakes or pieces, and introduced into the toothpaste base. The step of adding relatively insoluble flavorant into the slurry used to manufacture the hydratable, polymer matrix films typically requires the use of solvents such as ethanol. When making the films, the ethanol is typically removed using heat which causes the ethanol to evaporate. The low solubility flavorants are lost as a result of evaporation of the ethanol solvent. For examples, 50% of menthol in a slurry formula is lost with the solvent when the ethanol evaporates. Accordingly, introduction of low solubility flavorants into the film during its manufacture is inefficient, which leads to additional manufacturing costs.

There is a need for improved methods of manufacturing liquid, gel and semi-solid oral care products which comprise hydrophilic films containing menthol.

### SUMMARY

Methods of manufacturing dentifrices which comprise hydratable polymer matrix films that contain relatively water insoluble flavorants such as menthol are provided.

Provided is a method of preparing a dentifrice comprising polymer matrix film with low solubility flavorant therein comprising the steps of: forming a polymer matrix film comprising a water soluble polymer, a low solubility flavorant, and at least one material chosen from a low solubility polymer additive and a hydrophobic/lipophilic additive; providing a dentifrice base; and combining the polymer matrix film with the dentifrice base. Also provided are dentifrices formed from the methods.

### DETAILED DESCRIPTION

As used herein, the term “cellulose polymer” is meant to refer to cellulose and cellulose derivatives such as cellulose ester derivative and cellulose ether derivatives.

As used herein, the term “dentifrice” includes toothpastes and gels.

As used herein, such a “pharmaceutically acceptable” or “cosmetically acceptable” component is one that is suitable for use with humans and/or animals to provide the desired therapeutic, prophylactic, sensory, decorative, or cosmetic benefit without undue adverse side effects (such as toxicity, irritation, and allergic response) commensurate with a reasonable benefit/risk ratio.

As used herein, the term “polymer matrix film” is meant to refer to the product of a process wherein cellulose and derivatives thereof are used in combination with other polymers to form thin solid water hydratable film which may further comprise other components including, colloids and other particles. The polymer matrix film comprises one or more low solubility polymer additives. The polymer matrix film for example may further comprise additives such as, for example, colorants, water soluble flavorants, sweeteners, breath fresheners, whitening agents, and/or therapeutic agents such as agents that promote oral health, e.g. healthy teeth, gums and other oral tissue, and agents that prevent and treat various oral maladies. In addition, the polymer matrix film may include other film forming agents, plasticizing agents, surfactants and emulsifying agents. The polymer matrix film may be cut or otherwise divided into multiple pieces such as flakes or small strips and added to a dentifrice where they may provide aesthetic elements and/or serve as a carrier for one or more additives which may be included.

As used herein, the term “low solubility flavorant” refers to a flavor ingredient or cooling agent which is relatively insoluble in water, i.e. having the solubility generally on the order of menthol in water or less soluble at room temperature. A “low solubility flavorant” must first be incorporated into a solution using a solvent such as an alcohol, particularly ethanol, in order to stably incorporate it into the slurry of hydrophilic hydratable polymer which can be used to produce hydratable polymer matrix films comprising low solubility flavorants.

As used herein, the term “low solubility polymer additive” refers to a generally water insoluble polymer in which a low solubility flavorant is soluble. When a low solubility polymer additive is present in a polymer matrix film, the film has a greater capacity to undergo reverse migration of low solubility flavorant from a dentifrice base comprising flavorant to the polymer matrix film that comprises the copolymer additive. In addition or alternatively, when a low solubility polymer additive is present in a polymer matrix film, the film has a greater capacity to maintain a high concentration of when the polymer matrix comprising polymer is present in a dentifrice base comprising low solubility flavorant compared to concentration levels of when the polymer matrix comprising polymer is present in a dentifrice base comprising low solubility flavorant in a polymer matrix film that is free of polymer when such film is maintained in a dentifrice base comprising flavorant.

Throughout the present disclosure, ranges are used as shorthand for describing each and every value that is within the range. Any value within the range can be selected as the terminus of the range. Furthermore, all references cited throughout the disclosure are expressly incorporated by reference in their entireties. As used herein, all references to concentration of ingredients are on a weight basis, unless otherwise indicated.

Unless otherwise specified, all percentages and amounts expressed herein and elsewhere in the specification should be understood to refer to percentages by weight. The amounts given are based on the active weight of the material.

As described above, the films that are produced in US2008/0014224A1 to Boyd et al. result in a significant loss of menthol. This loss can be reduced to create a cost savings by including one or more low solubility polymer additives in the film and/or one or more hydrophobic/lipophilic additives. This low solubility polymer additive and/or the hydrophobic/lipophilic additive acts to retain more of the low solubility flavorant in the film. During use of the dentifrice, the films can be ruptured to release the low solubility flavorant to provide a more constant flavor profile.

Polymer matrix films provided herein comprise one or more species of water soluble polymers such as cellulose polymers, other polysaccharides and other polymers which are generally hydrophilic. Polymer matrix films may also comprise numerous other ingredients.

Typically, polymer matrix films comprise polymers present in an amount between 30% and 90% of the polymer matrix film's dry weight. The polymers may be present in an amount of between 40% and 80% of the polymer matrix film's dry weight. Some embodiments comprise polymers in an amount between 40% and 70% of the polymer matrix film's dry weight. Some embodiments comprise polymers an amount between 40% and 60% of the polymer matrix film's dry weight. Some embodiments comprise polymers an amount between 40% and 50% of the polymer matrix film's dry weight. Some embodiments comprise polymers in an amount between 50% and 80% of the polymer matrix film's dry weight. Some embodiments comprise polymers an amount between 60% and 80% of the polymer matrix film's dry weight. Some embodiments comprise polymers an amount between 65% and 75% of the polymer matrix film's dry weight.

Films useful for the present invention may be rigid or flexible, comprising any of a variety of materials, including film forming materials. In some embodiments, the film comprises at least one film-forming material, preferably comprising a polymer. Useful polymers include hydrophilic polymers, i.e. polymers soluble in a solvent, such as water. A water-soluble polymer that dissolves during exposure to water and application of physical force during use (such as during tooth brushing or scrubbing with a brush or pad) is desirable. Where the polymer does not fully break down during use, it may be a water-repellant polymer or an aqueous-stable hydrophilic polymer such as certain types of cellulose, e.g., paper. Examples of useful polymers are described in U.S. Pat. Nos. 4,713,243 to Schiraldi et al., 6,419,903, 6,419,906, 6,514,483 all to Xu, and 6,669,929 to Boyd et al.; United States Patent Publication Nos. 2004/0126332, 2004/0136924, and 2004/0042976 all to Boyd et al., and 2004/0062724 to Moro et al.

The polymer matrix film is hydratable and comprises a low solubility polymer additive and a low solubility flavorant. Additionally, the formulation of the polymer matrix films may be selected to affect release of active ingredient such as the amount released proportional to how vigorously or how long the composition is used, e.g., by brushing, scrubbing, or other mechanical action during use of the aqueous composition. The formulation of the polymer matrix films may be selected to produce an overall delayed and/or extended release of flavorant, thereby providing a flavor experience following product use.

Cellulose polymers are well known as is their use in water hydratable polymer matrix films. Cellulose polymers may be water soluble or water insoluble. Examples of cellulose derivatives include, but are not limited to: hydroxyalkyl methyl celluloses such as hydroxypropyl methyl cellulose, hydroxybutyl methyl cellulose, hydroxyethyl methyl cellulose, hydroxymethyl methyl cellulose and hydroxyethylpropyl methyl cellulose; carboxyalkyl methylcelluloses such as carboxypropyl methyl cellulose, carboxybutyl methyl cellulose, carboxyethyl methyl cellulose, carboxymethyl methyl cellulose and carboxyethylpropyl methyl cellulose; hydroxyalkyl celluloses such as hydroxypropyl cellulose, hydroxybutyl cellulose, hydroxyethyl cellulose, hydroxymethyl cellulose and hydroxyethylpropyl cellulose; alkyl celluloses such as propyl cellulose, butyl cellulose, ethyl cellulose (Ethocel™), methyl cellulose (Methocel™); and carboxyalkyl celluloses such as carboxypropyl cellulose, carboxybutyl cellulose, carboxyethyl cellulose, carboxymethyl cellulose and carboxyethylpropyl cellulose. Cellulose and cellulose ether derivative polymers may be of any length or combination of lengths. Moreover, the ranges of percent of substitutions may vary to ranges up to 100%. In molecules comprising two or more different substituting groups, the percentage substitution for each group is independent of the other groups.

The ratio of water soluble to low solubility cellulose polymers ranges from 10:1 to 5.5:2 for particularly useful polymer matrix films that provide good transfer characteristics of low solubility flavorants from base to polymer matrix film while providing good disintegration characteristics in aqueous liquid solutions. In some embodiments, the ratio of water soluble cellulose polymer to low solubility polymer that is incorporated in the polymer matrix film is 70:7, 69:9, 68:9, 67:10, 66:11, 65:12, 64:13, 63:14, 62:15, 61:16, 60:17, 59:18, 58:19, 57:20, 56:21. In some embodiments, the ratio of water soluble cellulose polymer to low solubility polymer that is incorporated in the polymer matrix film is 10:1, 9.75:1, 9.5:1, 9.25:1, 9:1, 8.75:1, 8.5:1, 8.25:1, 8:1, 7.75:1, 7.5:1, 7.25:1, 7:1, 6.75:1, 6.5:1, 6.25:1, 6:1, 5.75:1, 5.5:1, 5.25:1, 5:1, 4.75:1, 4.5:1, 4.25:1, 4:1, 3.75:1, 3.5:1, 3.25:1, 3:1 or 2.75:1. The cumulative amount of water soluble cellulose derivative and low solubility polymer that is incorporated in the polymer matrix film is typically 40%-90%, in some embodiments 50% to 80%. In some embodiments, the cumulative amount of water soluble cellulose derivative and low solubility polymer incorporated in the polymer matrix film is 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90%. In some embodiments, the cumulative amount of water soluble cellulose derivative and low solubility polymer incorporated in the polymer matrix film is 75% to 80%, and the ratio of water soluble cellulose polymer to low solubility polymer that is incorporated in the polymer matrix film is 70:7, 69:9, 68:9, 67:10, 66:11, 65:12, 64:13, 63:14, 62:15, 61:16, 60:17, 59:18, 58:19, 57:20, 56:21. In some embodiments, the cumulative amount of water soluble cellulose derivative and low solubility polymer incorporated in the polymer matrix film is 75% to 80%, and the ratio of water soluble cellulose polymer to low solubility polymer that is incorporated in the polymer matrix film is 10:1, 9.75:1, 9.5:1, 9.25:1, 9:1, 8.75:1, 8.5:1, 8.25:1, 8:1, 7.75:1, 7.5:1, 7.25:1, 7:1, 6.75:1, 6.5:1, 6.25:1, 6:1, 5.75:1, 5.5:1, 5.25:1, 5:1, 4.75:1, 4.5:1, 4.25:1, 4:1, 3.75:1, 3.5:1, 3.25:1, 3:1 or 2.75:1. In some embodiments, the cumulative amount of water soluble cellulose derivative and low solubility polymer incorporated in the polymer matrix film is 77%, and the ratio of water soluble cellulose polymer to low solubility polymer that is incorporated in the polymer matrix film is 70:7, 69:9, 68:9, 67:10, 66:11, 65:12, 64:13, 63:14, 62:15, 61:16, 60:17, 59:18, 58:19, 57:20, 56:21. In some embodiments, In some embodiments, the cumulative amount of water soluble cellulose derivative and low solubility polymer incorporated in the polymer matrix film is 77%, 78% or 79% and the ratio of water soluble cellulose polymer to low solubility polymer that is incorporated in the polymer matrix film is 10:1, 9.75:1, 9.5:1, 9.25:1, 9:1, 8.75:1, 8.5:1, 8.25:1, 8:1, 7.75:1, 7.5:1, 7.25:1, 7:1, 6.75:1, 6.5:1, 6.25:1, 6:1, 5.75:1; 5.5:1, 5.25:1, 5:1, 4.75:1, 4.5:1, 4.25:1, 4:1, 3.75:1, 3.5:1, 3.25:1, 3:1 or 2.75:1.

In some embodiments, the amount of low soluble polymer by weight is 33% or less. In some embodiments, the amount of low soluble polymer by weight is 32%, 31%, 30%, 29%, 28%, 27%, 26%, 25%, 24%, 23%, 22%, 21%, or less. In some embodiments, the amount of low soluble polymer by weight is 7% or more. In some embodiments, the amount of low soluble polymer by weight is 8%, 9%, 10%, 11%, 12%, 13%, 14%, 15%, 16%, 17%, 18%, 19%, or 20%. In other embodiments, the amount is 7 to 33 weight % or 11 to 21 weight %.

In some embodiments, the amount of water soluble cellulose polymer by weight is 70% or less. In some embodiments, the amount of water soluble cellulose polymer by weight is 56 or more. In some embodiments, the amount of water soluble cellulose polymer by weight is 69%, 68%, 67%, 66%, 65%, 64%, 63%, 62%, 61%, 60%, 59%, 58%, or 57%.

One or more species of water soluble polymers and one or more species of water insoluble polymers may be used to manufacture the polymer matrix films provided herein. An example of water insoluble cellulose polymers is ethyl cellulose. Typically, the ethyl cellulose used is from the family of ethyl cellulose products are available commercially from the Dow Chemical Company under the trade designation Ethocel™. An example of water soluble cellulose polymers is hydroxypropyl methyl cellulose (HPMC). Typically, HPMC used is from the family of HPMC and methyl cellulose (MC) products are available commercially from the Dow Chemical Company under the trade designation Methocel™. The ratio of HPMC and ethyl cellulose ranges from 10:1 to 5.5:2 for particularly useful polymer matrix films that provide good transfer characteristics of low solubility flavorants from base to polymer matrix film while providing good disintegration characteristics in aqueous liquid solutions. In some embodiments, the ratio of Methocel™ (for example Methocel™ E5) to Ethocel™ (for example Ethocel™ S4) that is incorporated in the polymer matrix film is 70:7, 69:9, 68:9, 67:10, 66:11, 65:12, 64:13, 63:14, 62:15, 61:16, 60:17, 59:18, 58:19, 57:20, 56:21. In some embodiments, the ratio of Methocel™ (for example Methocel™ E5) to Ethocel™ (for example Ethocel™ S4) that is incorporated in the polymer matrix film is 10:1, 9.75:1, 9.5:1, 9.25:1, 9:1, 8.75:1, 8.5:1, 8.25:1, 8:1, 7.75:1, 7.5:1, 7.25:1, 7:1, 6.75:1, 6.5:1, 6.25:1, 6:1, 5.75:1, 5.5:1, 5.25:1, 5:1, 4.75:1, 4.5:1, 4.25:1, 4:1, 3.75:1, 3.5:1, 3.25:1, 3:1 or 2.75:1. In some embodiments, the cumulative amount of Methocel™ (for example Methocel™ E5) to Ethocel™ (for example Ethocel™ S4) incorporated in the polymer matrix film is 41%, 42%, 43%, 44%, 45%, 46%, 47%, 48%, 49%, 50%, 51%, 52%, 53%, 54%, 55%, 56%, 57%, 58%, 59%, 60%, 61%, 62%, 63%, 64%, 65%, 66%, 67%, 68%, 69%, 70%, 71%, 72%, 73%, 74%, 75%, 76%, 77%, 78%, 79%, 80%, 81%, 82%, 83%, 84%, 85%, 86%, 87%, 88%, 89% or 90% by weight. These amounts and ratios are particularly useful polymer matrix films that provide good transfer characteristics of low solubility flavorants from base to polymer matrix film while providing good disintegration characteristics in aqueous liquid solutions. In some embodiments, the cumulative amount of HPMC, such as Methocel™ (for example Methocel™ E5), and ethyl cellulose, such as Ethocel™ (for example Ethocel™ S4), incorporated in the polymer matrix film is 75% to 80%, and the ratio of HPMC, such as Methocel™ (for example Methocel™ E5), to ethyl cellulose, such as Ethocel™ (for example Ethocel™ S4), that is incorporated in the polymer matrix film is 10:1, 9.75:1, 9.5:1, 9.25:1, 9:1, 8.75:1, 8.5:1, 8.25:1, 8:1, 7.75:1, 7.5:1, 7.25:1, 7:1, 6.75:1, 6.5:1, 6.25:1, 6:1, 5.75:1, 5.5:1, 5.25:1, 5:1, 4.75:1, 4.5:1, 4.25:1, 4:1, 3.75:1, 3.5:1, 3.25:1, 3:1 or 2.75:1. In some embodiments, the cumulative amount of HPMC, such as Methocel™ (for example Methocel™ E5), and ethyl cellulose, such as Ethocel™ (for example Ethocel™ S4), incorporated in the polymer matrix film is 77%, and the ratio of HPMC, such as Methocel™ (for example Methocel™ E5), to ethyl cellulose, such as Ethocel™ (for example Ethocel™ S4), that is incorporated in the polymer matrix film is 70:7, 69:9, 68:9, 67:10, 66:11, 65:12, 64:13, 63:14, 62:15, 61:16, 60:17, 59:18, 58:19, 57:20, 56:21. In some embodiments, In some embodiments, the cumulative amount of HPMC, such as Methocel™ (for example Methocel™ E5), and ethyl cellulose, such as Ethocel™ (for example Ethocel™ S4), incorporated in the polymer matrix film is 77%, 78% or 79% and the ratio of HPMC, such as Methocel™ (for example Methocel™ E5), to ethyl cellulose, such as Ethocel™ (for example Ethocel™ S4), that is incorporated in the polymer matrix film is 10:1, 9.75:1, 9.5:1, 9.25:1, 9:1, 8.75:1, 8.5:1, 8.25:1, 8:1, 7.75:1, 7.5:1, 7.25:1, 7:1, 6.75:1, 6.5:1, 6.25:1, 6:1, 5.75:1, 5.5:1, 5.25:1, 5:1, 4.75:1, 4.5:1, 4.25:1, 4:1, 3.75:1, 3.5:1, 3.25:1, 3:1 or 2.75:1.

In some embodiments, the amount of Ethocel™ (for example Ethocel™ S4) by weight is 21% or less. In some embodiments, the amount of Ethocel™ (for example Ethocel™ S4) by weight is 11% or more. In some embodiments, the amount of Ethocel™ (for example Ethocel™ S4) by weight is 12%, 13%, 14%, 15%, 16%, 17%, 18%, 19%, or 20%.

In some embodiments, the amount of Methocel™ (for example Methocel™ E5) by weight is 70% or less. In some embodiments, the amount of Methocel™ (for example Methocel™ E5) by weight is 56 or more. In some embodiments, the amount of Methocel™ (for example Methocel™ E5) by weight is 69%, 68%, 67%, 66%, 65%, 64%, 63%, 62%, 61%, 60%, 59%, 58%, or 57%.

Polymer matrix films that may comprise hydrophobic/lipophilic additives include compounds that can be incorporated into the polymer matrix films, particularly during manufacture and which, when in incorporated as part of a polymer matrix film, can serve to attract low solubility flavorants from the dentifrice base and concentrate them to the polymer matrix film. Illustrative examples of hydrophobic/lipophilic additives suited for inclusion into the composition include, fats and oil including but not limited to, petrolatum, silicone oil, beeswax, hydrogenated soybean oil, sweet almond oil, peanut oil, avocado oil, borage oil, palmitic acid, cacao butter, carnauba wax, castor oil, coconut oil, evening primrose oil, glycerin, glyceryl stearate, jojoba oil, camphor, kkaolin, lanolin, cod liver oil, linseed oil, corn oil, olive oil, palm oil, paraffin, squalane, rapeseed oil, rose oil, safflower oil, sesame oil, shea butter, dimethicone silicone oil, tall oil, wheat germ oil, sunflower oil, trimethylsiloxysilicate, alkyldimethylsilyl solypropylsesquioxane, dimethiconol, trimethylsiloxysilicate, polyether-modified silicone, cross-linked polymers, polypropylsilsesquioxane, dimethicone polymers, dimethicone crosspolymer, dimethicone/vinyl dimethicone cross polymers, other fats and oils, and combinations thereof.

The hydrophobic/lipophilic additives may be present in an amount of between 1% and 20% of the polymer matrix film's dry weight. Some embodiments comprise hydrophobic/lipophilic additives in an amount between 3% and 15% of the polymer matrix film's dry weight. Some embodiments comprise hydrophobic/lipophilic additives an amount between 5% and 10% of the polymer matrix film's dry weight. Some embodiments comprise hydrophobic/lipophilic additives an amount between 1% and 8% of the polymer matrix film's dry weight.

In some embodiments, polymer matrix films comprise colloids. The colloid may present in an amount between 10% and 60% of the polymer matrix film's dry weight. The colloid may present in an amount between 20% and 50% of the polymer matrix film's dry weight. The colloid may present in an amount between 30% and 50% of the polymer matrix film's dry weight. The colloid may present in an amount between 40% and 50% of the polymer matrix film's dry weight.

Colloids and colloidal particles can be used to stabilize polymer matrices and fine tune its rigidity in order to provide films that are flexible enough to process, yet physically and cosmetically stable. As films are optimized, it is important to identify the parameters that will deliver optimal film performance. These parameters can be determined by quantifying the properties of the film at both the slurry stage and the dry film stage. At the slurry stage, the interactions between the polymers and the other film ingredients, including colloidal particles, form the structure of the film matrix. The viscoelastic properties of the slurry, such as the viscosity and the structural parameter (G′), enable the characterization of structural arrangement within the slurry and the processability of the same. Following processing and drying of the slurry, the bulk film is formed, setting the polymer matrix. Mechanical properties, such as the glass transition temperature, the tensile strength, and the dissolution time can be used to determine the stability of the film. By balancing the microstructural properties, such as the polymer interactions, with the macrostructural properties of the film, such as the mechanical properties, film can be made more cosmetically stable and can be better utilized as a delivery platform for various actives.

In some embodiments, colloidal particles are present in the film in the range of 40-50% dry weight.

Water-insoluble colloidal metal compounds of multivalent metals are preferred. Representative metal oxides suitable for use in the compositions described herein include silicon oxide (SiO2), molybdenum oxide (Mo₂O₃), aluminum oxide (Al₂O₃), titanium oxide (TiO), zirconium oxide (ZrO₂) and zinc oxide (ZnO).

Particle size may be 1 to 1000 nm. Preferably the particles have an average particle size of 1 μm to 850 nm, 50 μm to 150 nm, 15 nm to 500 nm, 30 nm to 250 nm and/or 5 μm to 100 nm.

In some embodiments, the particles are non-aggregated. By non-aggregated it is meant that the particles are not massed into a cluster having a size greater than 1 micron, preferably greater than 950 nm or 850 nm. However, particles may be mixed with aggregated particles and other colloidal particles that have an average particle size of greater than 1 micron if desired. In some embodiments, more than 80% of particles are non-aggregated. In some embodiments, more than 90% of particles are non-aggregated.

In some embodiments, colloidal particles are provided in the dentifrice base. In some embodiments, colloidal particles are provided in the dentifrice base and the polymer matrix film. In some embodiments, colloidal particles are provided in the dentifrice base but not the polymer matrix film.

In preparing the film matrix, the water soluble polymers, the low solubility polymer, the low solubility flavorant, and any of the optional ingredients, including for example, such as those set forth below as “Other Components”, are dissolved or otherwise mixed into a compatible solvent to form a film forming composition. The film forming composition may contain no flavorant and no flavor solvent. The film forming composition is cast on a releasable carrier and dried to form a sheet of film matrix material. In some embodiments, the carrier material has a surface tension which allows the film solution to spread evenly across the intended carrier width without soaking to form a destructive bond between the film carrier substrates. Examples of suitable carrier materials include glass, stainless steel, Teflon and polyethylene-impregnated paper. Drying of the film may be carried out at high temperature using a drying oven, drying terminal, vacuum drier, or any other suitable drying equipment which does not adversely affect the ingredients of which the film is composed.

The slurries that are precursors to the films may be characterized using rheology. In some embodiments, the viscoelastic properties of the film slurry, as quantified using G′ as an indicator of the structural character of the polymer-particle network, may be 220-560. In some embodiments G′ is 223-550. In some embodiments, the structure of the polymer-particle matrix is not weak and the slurry is not essentially liquid-like. In some embodiments, the structure of the polymer-particle matrix is not very rigid thereby not leading to the formation of a very brittle film. In some embodiments, the viscosity profile as a function of shear rate is quantified as a measure of flowability and processability the slurries. In some embodiments, the viscosity profiles are not a semi-dilute solution. The viscosity in poise is measured at 0.3 s-1. In some embodiments, the viscosity (taken at 0.3 s-1) for the various slurries is 175-475. In some embodiments, the viscosity (taken at 0.3 s-1) for the various slurries is 183-450.

The films of the present invention preferably have a substantially lamellar structure. A “lamellar” structure has a size in one or two dimensions (e.g., x- or y-dimensions) that is substantially greater than the thickness of the structure in a third dimension (e.g., the z-dimension), and generally includes substantially planar, layered, or lamelliform shapes, for example. In one embodiment, the lamellar structure is substantially planar, having a size in both the x- and y-dimensions that is substantially greater than the z-dimension. In other embodiments, the lamellar structure is non-planar. In one embodiment, a film comprises a substantially continuous surface that can appear as a substantially flat surface, although in some embodiments the film may be deformed. In such embodiments, the film can have any of a number of shapes, including having a smooth, curved surface. Further, the term “film” encompasses both a single structure as well as a plurality of film fragments. In certain embodiments, the film comprises a plurality of fragments independently having a thickness of 0.1 mils to 10 mils, preferably 0.5 mils to 9 mils, and more preferably 1.2 mils to 3 mils. In some embodiments, the film thickness range is 2 to 3 microns. A preferred length of the fragments is at least 0.2 mm.

The dried film is then processed for inclusion in the dentifrice. The film may be cut or punched into small strips or squares. In various embodiments, the film comprises a plurality of fragments or pieces. Such fragments may be of any of a variety of shapes or forms, including semi-solid or solid discrete portions, fragments, particles, flakes, or mixtures thereof. In various embodiments, the film fragments have a recognizable shape. In some embodiments, a film fragment comprises a nonrandom shape. Such shapes include simple geometric shapes such as polygons, elliptical shapes, triangles, quadrilaterals (such as a square, a rectangle, a rhombus), pentagons, hexagons, ovals, circles, or shapes that are representative of figures, animate or inanimate objects, such as stars, hearts, gems, flowers, trees, shamrocks, letters, numbers, animals, characters, diamonds, circles and the like. The dried film may be cut or punched into shaped flakes having a particle size of 0.01 to 0.50 inches preferably 0.08 to 0.25 inches. Additional stability can be provided to the shapes formed from the dried film, by applying to the film, before shaping into flakes or small strips, a protective barrier overcoat such as a food grade shellac or ethyl cellulose.

Further, the plurality of film fragments may have different compositions, for example having a first plurality of film fragments comprising a first color, and a second plurality of film fragments comprising a second color, where the first and second colors are different from each other. Any permutation of different compositions is contemplated, for example, any number of different active ingredients in the compositions or different film compositions.

Examples of suitable carriers for oral care compositions are disclosed in U.S. Pat. Nos. 6,669,929 to Boyd et al., 6,379,654 to Gebreselassie et al., and 4,894,220 to Nabi et al. The dentifrice (toothpaste or gel) is typically water based. As recognized by one of skill in the art, the dentifrice optionally include other materials and mixtures thereof, including for example, such as those set forth below as “Other Components”. It is understood that while general attributes of each of the above categories of materials may differ; there may be some common attributes, and any given material may serve multiple purposes within two or more of such categories of materials.

In the preparation of the base dentifrice in accordance with the present invention there is utilized an orally acceptable vehicle, including a water-phase with humectants. Humectants useful herein include polyhydric alcohols such as glycerin, sorbitol, xylitol or low molecular weight PEGs, alkylene glycol such as polyethylene glycol or propylene glycol. In various embodiments, humectants are operable to prevent hardening of paste or gel compositions upon exposure to air. In various embodiments humectants also function as sweeteners. One or more humectants are optionally present in a total amount of 1% to 50%, for example 2% to 25% or 5% to 15%. Humectants are present typically in amount of 5 to 10% by weight in water, typically, 30 to 80% by weight of the dentifrice, more typically 50 to 70% by weight.

The base dentifrice may also contain an inorganic or a natural or synthetic thickener or gelling agent. Optionally, one or more thickening agents are optionally present in a total amount of 0.01% to 15%, in some embodiments 0.1% to 10%, in some embodiments 0.10 to 5% by weight, in some embodiments 0.2% to 5% by weight and in some embodiments 0.2 to 1% by weight. These proportions of thickeners in the dentifrice compositions of the present invention in which the film flakes of the present invention are suspended are sufficient to form an extrudable, shape-retaining product which can be squeezed from a tube onto a toothbrush and will not fall between the bristles of the brush but rather, will substantially maintain its shape thereon. Suitable thickeners or gelling agents useful in the practice of the present invention include inorganic thickening silicas such as amorphous silicas available from Huber Corporation under the trade designation Zeodent 165, Irish moss, iota-carrageenan, polyvinylpyrrolidone, carboxyvinyl polymers, cellulosic polymers such as hydroxyethylcellulose, carboxymethylcellulose (carmellose) and salts thereof (e.g., carmellose sodium), natural gums such as karaya, xanthan, gum arabic and tragacanth, colloidal magnesium aluminum silicate, colloidal silica and mixtures thereof.

In various embodiments, a dentifrice composition is provided within a single component or phase. In other embodiments, the composition includes both a first and a second component that are separately maintained. Maintaining the components separately requires only that the components are maintained in such a way as to substantially prevent the interaction of one component of the composition with another component of the composition. Typically, a dual component oral care composition is employed where there are one or more incompatible ingredients included in the composition. For example, if the dentifrice comprises two incompatible active ingredients, it is advantageous to maintain them separately. While the films comprising active ingredients generally provide a degree of separation, there may be some migration of active from the film into the carrier, and vice versa, and as such, in some cases it may desirable to provide an entirely separate phase. The separation of components can be accomplished through any means known or to be discovered in the art and includes chemical, physical, and mechanical means of separation of any combination of these. For example, the first and second incompatible components may be combined but certain components are separately maintained by wrapping or encapsulating one or both in a protective film, coating, capsule, micelle, etc.

Optionally, the low solubility flavorant can be present in the dentifrice base in concentrations of 0.025-10% by weight. Typically, low solubility flavorant is present in the base at a concentration of 0.05 to 7.5% based on the total weight. In some embodiments, low solubility flavorant is present in a concentration of 0.1 to 5% by weight, in some embodiments, 0.5 to 2.5% by weight, in some embodiments, 0.75 to 2% by weight, in some embodiments, 1.0 to 1.5% by weight.

Typically, to prepare the dentifrice base, water, humectants, e.g. glycerin, sorbitol polyethylene glycol are dispersed in a conventional mixer until the mixture becomes a homogeneous gel phase. Into the gel phase are added other ingredients and mixed until a homogeneous phase is obtained. Thereafter the thickener, any flavor and surfactant ingredients are added and the ingredients mixed at high speed until vacuum of 20 to 100 mmHg.

In some embodiments, the dentifrice base comprises one or more other components selected from the group consisting of: polyethylene glycol, CMC, sodium saccharin, sodium fluoride, sorbitol (70% solution), purified water, colorant, silica zeodent, cocaamidopropyl betaine and sodium lauryl sulfate.

Menthol is contemplated to be the preferred low solubility flavorant. In addition to menthol, other low solubility flavor ingredients or cooling agents, natural or synthetic, may be incorporated into polymer matrix films using in-situ flavoring of films produced free of low solubility flavorants by adding the films produced free of low solubility flavorants into any toothpaste base which comprises the low solubility flavorants.

Flavor agents are known, such as natural and artificial flavors. These flavorants may be chosen from synthetic flavor oils and flavoring aromatics, and/or oils, oleo resins and extracts derived from plants, leaves, flowers, fruits and so forth, and combinations thereof. In addition to menthol, representative flavor oils include: spearmint oil, cinnamon oil, peppermint oil, clove oil, bay oil, thyme oil, cedar leaf oil, oil of nutmeg, oil of sage, and oil of bitter almonds. These flavor agents can be used individually or in admixture. Commonly used flavors include mints such as peppermint, artificial vanilla, cinnamon derivatives, and various fruit flavors, whether employed individually or in admixture. To be considered low solubility flavorants, the flavorants may be hydrophobic, insoluble or must be sufficiently insoluble in water so that they must be solubilized in a solvent such as ethanol or another alcohol in order to incorporate them into a slurry that can be used to produce a polymer film matrix at a practical level for use as a flavorant.

The low solubility flavorants may be present in the film in any desired amount, such as 0.01 to 10% by weight. The low solubility flavorants may also be included in the dentifrice in any desired amount, such as 0.01 to 10% by weight.

Additional components may also be included in the dentifrice base and/or the polymer matrix films. In some embodiments, one or more additional components are provided in both the dentifrice base and the polymer matrix film. In some embodiments, one or more additional components are provided are provided in the dentifrice base but not the polymer matrix film. In some embodiments, one or more additional components are provided in the polymer matrix film but not in the dentifrice base.

Preferably, the polymer matrix film and/or the dentifrice base optionally comprises one or more of the following additional components: surface active agents, bulking agents, viscosity modifiers, surfactants, thickeners, humectants, diluents, fillers (in addition to those described above), pH modifying agents, plasticizers, fillers, waxes, texture modifiers, oils, flavoring and/or sweetening agents, colorants, dyes, whitening agents, breath freshening agents, abrasives, polishing agents, preservatives, solvents, and mixtures thereof. In embodiments prophylactic and therapeutic agents such as: cetylpyridinium chloride, chlorhexidene, fluoride ion sources, stannous ion sources, tartar control (anticalculus) agents, antimicrobial (e.g., antibacterial) agents, antioxidants, saliva stimulating agents, antiplaque (e.g., plaque disrupting) agents, anti-inflammatory agents, H2 antagonists, desensitizing agents, nutrients, proteins and combinations and mixtures thereof. It is understood that while general attributes of each of the above categories of materials may differ; there may be some common attributes, and any given material may serve multiple purposes within two or more categories of materials.

### Dentifrice Composition Comprising Dentifrice Base and Polymer Matrix Films

The film flakes and strips made from the polymer matrix film that comprise a low solubility polymer are incorporated in the base dentifrice of the present invention, preferably at a concentration of 0.05 to 1.0% by weight and preferably 0.1 to 0.5% by weight. The film flakes or strips are generally added to the dentifrice base as a last step, so as to minimize the shear to which the dentifrice ingredients are subjected to during the prior mixing steps.

In some embodiments, the film matrix is rupturable during tooth brushing so that one or more additives such as the low solubility flavorant is released when the dentifrice is applied topically to tooth surfaces, the mechanical agitation created during tooth brushing effecting rupture of the film matrix whereby the entrained ingredient is released to the tooth surface. In some embodiments, the complete release is extended such that the flavor experience continues after the oral care procedure is performed.

### EXAMPLES

### Example 1

Table 1 (below) describes the ingredients of film slurries with and without the polyvinylpyrrolidone/vinyl acetate copolymer.

TABLE 1

| Ingredient | Comparative film (%) | Co-polymer film (%) |
|---|---|---|
| Water | 68.2 | 64.2 |
| Methocel E5 | 19 | 19 |
| PVP/VA E-335* | — | 4 |
| Saccharin | 1 | 1 |
| Propylene Glycol | 4 | 4 |
| Colorant | 0.03 | 0.03 |
| Tween 80 | 0.8 | 0.8 |
| Flavor | 7 | 7 |

*50% of Vinylpyrrolidone/vinyl acetate copolymer solution

Table 2 (below) describes the ingredients of dried films with and without the polyvinylpyrrolidone/vinyl acetate copolymer.

TABLE 2

| | Comparative Film | Co-polymer film |
|---|---|---|
| Ingredient | (wt. %) | (wt. %) |
| Water | 2 | 2 |
| Methocel E5 | 60.1 | 55.4 |
| PVP/VA copolymer | — | 5.8 |
| Saccharin | 3.2 | 2.9 |
| Propylene Glycol | 12.7 | 11.7 |
| Colorant | 0.09 | 0.09 |
| Flavor | 19.4 | 19.8 |
| Tween 80 | 2.5 | 2.3 |

Table 3 (below) describes the toothpaste base into which the films of the present invention and the comparative films were included in the evaluation described below.

TABLE 3

| Ingredient | Toothpaste Base (wt. %) |
|---|---|
| Polyethylene Glycol 600 | 1 |
| CMC | 0.55 |
| Sodium Saccharin | 0.35 |
| Sodium Fluoride | 0.32 |
| Sorbitol (70% solution) | 68 |
| Water | 9.05 |
| Colorant | 0.01 |
| Zeodent 114 | 8 |
| Zeodent 165 | 8 |
| Cocamidopropyl Betaine | 1.25 |
| Sodium Lauryl Sulfate | 1.57 |

### Example 2

Test films were cut into circular discs of 0.25 inches diameter using a punch. Twenty four pieces of each film were used for the investigation. Test toothpaste was made by mixing base toothpaste (as described in Table 3, above) with film discs (those of the present invention and the comparative films) at the ratio of 98.1/0.5.

After two, four and eight weeks of aging at room temperature, four discs of the films of the present invention and the comparative films were isolated from the aged test toothpaste. Toothpaste was removed from the film discs with a spatula; with the remaining toothpaste being rubbed off four times with tissue. The film discs were then dissolved in 10 ml of Acetonitrate/Water (80/20) in a 20 ml vial. The concentration of flavor (Iralia) present in the solution was quantified using HPLC. The percentage of migrated flavor was then calculated based on the concentration of flavor found in the solution. The results are summarized in Table 4 (below).

TABLE 4

| Film | Two weeks | Four weeks | Eight weeks |
|---|---|---|---|
| Comparative film | 30.1% | 53.8% | 61.9% |
| Co-polymer film | 2.9% | 3.4% | 43.5% |

The data described in Table 4 (above) illustrates that films of the present invention provide improved flavor stability.

What is claimed is:

1. A method of preparing a dentifrice comprising a polymer matrix film with low solubility flavorant contained therein comprising the steps of:
a) forming a polymer matrix film comprising a water soluble polymer, a low solubility flavorant, and at least one material chosen from a low solubility polymer additive and a hydrophobic/lipophilic additive;
b) providing a dentifrice base; and
c) combining the polymer matrix film with the dentifrice base.

2. The method of claim 1, wherein the low solubility flavorant is selected from the group consisting of: menthol, spearmint oil, cinnamon oil, peppermint oil, clove oil, bay oil, thyme oil, cedar leaf oil, oil of nutmeg, oil of sage, and oil of bitter almond, said water soluble polymer comprises hydroxypropyl methyl cellulose and the low solubility polymer additive is ethyl cellulose.

3. (canceled)

4. (canceled)

5. (canceled)

6. The method of claim 1, wherein the combined water soluble polymer and low solubility polymer additive makes up by weight 40 to 90% of polymer film matrix, and the ratio of water soluble polymer to low solubility polymer additive by weight is 10:1 and 2.75:1.

7. The method of claim 1, wherein the amount of low solubility polymer additive in the polymer matrix film is 7% to 33% by weight, or 11% to 21% by weight and the amount of water soluble polymer in the polymer matrix film is 56% to 70% by weight or 56% to 66% by weight.

8. (canceled)

9. The method of claim 1, wherein the hydrophobic/lipophilic additive is selected from the group consisting of: petrolatum, silicone oil, beeswax, hydrogenated soybean oil, sweet almond oil, peanut oil, avocado oil, borage oil, palmitic acid, cacao butter, carnauba wax, castor oil, coconut oil, evening primrose oil, glycerin, glyceryl stearate, jojobaoil, camphor, kaolin, lanolin, cod liver oil, linseed oil, corn oil, olive oil, palm oil, paraffin, squalane, rapeseed oil, rose oil, safflower oil, sesame oil, shea butter, dimethicone silicone oil, tall oil, wheat germ oil, sunflower oil, and combinations thereof.

10. The method of claim 1, wherein the polymer matrix film further comprises zinc oxide.

11. The method of claim 1, wherein the polymer matrix film further comprises one or more additional components selected from the group consisting of: diols, surfactants, starches, colorants, dyes, sweeteners, whitening agents, breath freshening agents, abrasives, cationic prophylactic and therapeutic agents, fluoride ion sources, stannous ion sources, tartar control agents, antimicrobial agents, antioxidants, saliva stimulating agents, antiplaque agents, anti-inflammatory agents, H2 antagonists, desensitizing agents, nutrients, and proteins; and
wherein the dentifrice base further comprises one or more additional components selected from the group consisting of: flavorants, low solubility flavorants, diols, surfactants, starches, colorants, dyes, sweeteners, whitening agents, breath freshening agents, abrasives, cationic prophylactic and therapeutic agents, fluoride ion sources, stannous ion sources, tartar control agents, antimicrobial agents, antioxidants, saliva stimulating agents, antiplaque agents, anti-inflammatory agents, H2 antagonists, desensitizing agents, nutrients, and proteins.

12. (canceled)

13. The method of claim 1 further comprising the steps of:
forming a polymer matrix film by forming a slurry comprising the water soluble polymer, the low solubility polymer additive, and the low solubility flavorant,
dispensing the slurry on a surface wherein the slurry forms a layer of slurry on the surface, and drying the layer of slurry to produce the polymer matrix film.

14. The method of claim 13, further comprising the step of after drying the slurry layer to form the polymer matrix film, cutting or punching the polymer matrix film to form film flakes or strips of polymer matrix film prior to combining the film with the dentifrice base.

15. The method of claim 1, wherein the polymer matrix film that is formed comprises Methocel E5, Ethocel S4, Propylene Glycol and Tween 80.

16. The method of claim 1, wherein polymer matrix film is formed in the substantial absence of alcohol.

17. The method of claim 1, wherein the dentifrice base that is provided comprises: Polyethylene Glycol 600, CMC 50T, Sodium Saccharin, Sodium Fluoride, Sorbitol, Purified Water, D&C Red No. 30, Silica Zeodent 114, Silica Zeodent 165, Cocaamidopropyl Betaine, and Sodium Lauryl Sulfate.

18. The method of claim 1, wherein the combined polymer matrix film and dentifrice base comprises 0.2% menthol.

19. The method of claim 1, wherein the combined polymer matrix film and dentifrice base comprises 1% polymer matrix film.

20. A dentifrice product produced by the method of claim 1.

21. The method of claim 1, wherein the polymer matrix film comprises a polyvinylpyrrolidone/vinyl acetate (PVP/VA) copolymer.

22. The method of claim 1, wherein less than about 5% of the low solubility flavorant migrates out of said polymer matrix film after 4 weeks at room temperature.

23. The method of claim 21, wherein the PVP/VA copolymer is present at a concentration of from about 4 wt. % to about 7 wt. %, of the dried film.

24. The method of claim 23, wherein the PVP/VA copolymer is present at a concentration of from about 5 wt. % to about 6 wt. %, of the dried film.

25. The method of claim 2, wherein
the combined water soluble polymer and low solubility polymer additive makes up by weight 40 to 90% of polymer film matrix,
the ratio of water soluble polymer to low solubility polymer additive by weight is 10:1 and 2.75:1 and
the hydrophobic/lipophilic additive is selected from the group consisting of: petrolatum, silicone oil, beeswax, hydrogenated soybean oil, sweet almond oil, peanut oil, avocado oil, borage oil, palmitic acid, cacao butter, carnauba wax, castor oil, coconut oil, evening primrose oil, glycerin, glyceryl stearate, jojobaoil, camphor, kaolin, lanolin, cod liver oil, linseed oil, corn oil, olive oil, palm oil, paraffin, squalane, rapeseed oil, rose oil, safflower oil, sesame oil, shea butter, dimethicone silicone oil, tall oil, wheat germ oil, sunflower oil, and combinations thereof.
